(19) United States
(12) Patent Application Publication
(10) Pub. No.: US 2003/0205077 A1
(43) Pub. Date: Nov. 6, 2003

(54) Continuous on-line carbon analyzer

(76) Inventor: Stephen Staphanos, Long Beach, CA (US)

Correspondence Address: Christopher R. Christenson, WESTMAN CHAMPLIN & KELLY, Suite 1600 - International Centre, 900 South Second Avenue, Minneapolis, MN, 55402-3319, US

(21) Appl. No.: 10/000,187

(22) Filed: Oct. 18, 2001

(51) Int. Cl. G01N 33/18; G01N 33/00

(52) U.S. Cl. 73/19.1; 73/61.41

(57) ABSTRACT

A continuous on-line combustion-type carbon analyzer for water applications is provided. The analyzer receives a continuous stream of sample and carbon dioxide-free gas. The analyzer includes control components to limit sample flow into a combustion furnace such that excessive pressures and thermal stresses are minimized. The sample specimen is oxidized within the combustion furnace and the oxidized sample is conveyed to a detector that provides a continuous read-out of carbon quantity in the sample stream.

### FIELD OF THE INVENTION

The present invention is related to carbon analysis. More specifically, the present invention is related to a system for continuously measuring carbon content in an aqueous sample stream in substantially real-time.

### BACKGROUND OF THE INVENTION

Analysis of both organic and inorganic carbon content provides valuable information for a number of water processing applications. For example, such information can provide valuable insight regarding the efficacy of raw water management during waste water processing, monitoring of cooling water, cleaning water, purified water, as well as a monitoring of waste water effluent. Many pollutants and other undesirable substances generally contain at least some form of carbon. Thus, monitoring carbon content can provide an indication of the presence and quantitative nature of such substances. Such information can be a valuable diagnostic tool for monitoring, or otherwise controlling water treatment and processing facilities.

In general, there are two primary types of carbon analysis that are currently performed. The goal of both such types of analysis is to fully oxidize all carbon present in the sample into carbon dioxide and subsequently detect the relative amount of carbon dioxide. A common method in which carbon dioxide is detected is using a non-dispersive infrared (NDIR) detector. The two types of analyses differ in how oxidation is effected.

The first type of carbon analysis is known as low-temperature analysis and generally is performed at temperatures at or below 100° C. One example of such low temperature oxidation is the utilization of ultraviolet irradiation to bombard the sample, and with sufficient exposure, oxidize all dissolved organics into CO₂. Carbon dioxide, in this case, can also be detected by measuring a change in conductivity of the sample. Another type of low-temperature carbon analysis utilizes a heated persulfate solution. In general, a sample is mixed with a quantity of persulfate solution and heated to approximately 100° C. After a pre-selected interval, the resulting CO₂ is purged out by a carrier gas and detected by an non-dispersive infrared (NDIR) sensor. Both of the above analyses generally require significant time in order to realize complete oxidation of the sample. A third low-temperature technique combines the above two techniques and uses a persulfate solution in addition to UV radiation. Thus, the sample is simultaneously exposed to persulfate and UV radiation. The resulting carbon dioxide is purged out by a carrier gas and detected by an NDIR sensor. Oxidation is more vigorous than the above methods and thus provides faster analysis.

One of the drawbacks of low-temperature analyses, also know as wet-chemical oxidation, is that particulate matter is somewhat difficult to deal with. Particulates, by their nature, are usually more difficult to oxidize and some organics may escape exposure to UV agents by being positioned within the interstitial spaces of the particles.

High-temperature techniques, also known as combustion techniques, generally expose the specimen to a high-temperature. Additionally, it is common to use a catalyst in order to facilitate more effective oxidation. One particular combustion technique utilizes a platinum-based catalyst and a combustion temperature in excess of approximately 680° C. Carbon-containing specimens are fully oxidized to carbon dioxide under the above conditions. The resultant carbon dioxide is provided to a detector, generally an NDIR detector, for further analysis. High-temperature carbon analysis provides an advantage in that oxidation can be effected relatively quickly compared to low-temperature techniques. Further, specimens that are difficult to oxidize via low-temperature techniques are readily oxidizable with high-temperature techniques.

One of the difficulties of using high-temperature analyses, or combustion techniques, for carbon analysis is the relatively severe temperature changes that the sample undergoes during processing. A relatively small amount of liquid sample can become a significant amount of steam and carbon dioxide. Additionally, the thermal shock upon the combustion chamber can be significant as a specimen is introduced at a relatively low temperature and quickly heated by the combustion chamber to combustion temperatures. In general, therefore, combustion techniques are performed in a batch mode. In such a system, a pre-selected amount of sample is conveyed to the combustion chamber to ensure that the thermal mass and resultant gas and steam do no overly stress the system. However, batch-processing introduces a temporal lag that can adversely effect real-time control of water processing.

One device that appears to provide on-line measurement of carbon content in water is commercially available from Shimadzu products under the trade designation TOC-4000. The product information for this device provides for a measurement cycle of approximately four minutes thus indicating a batch process that is performed successively. As stated above, batch processing introduces a temporal lag for real-time control of data processes. Additionally, the output from a detector in such a system would include undesirable peaks rendering data proximate the peak unusable. Therefore, there is a continuing need for a real-time carbon analyzer for water processing applications.

### SUMMARY OF THE INVENTION

A continuous on-line combustion-type carbon analyzer for water applications is provided. The analyzer receives a continuous stream of sample and carbon dioxide-free gas. The analyzer includes control components to limit sample flow into a combustion furnace such that excessive pressures and thermal stresses are minimized. The sample specimen is oxidized within the combustion furnace and the oxidized sample is conveyed to a detector that provides a continuous read-out of carbon quantity in the sample stream.

### BRIEF DESCRIPTION OF THE DRAWINGS

FIG. 1. is a diagrammatic view of an on-line continuous combustion-type carbon analyzer for water applications.

FIG. 2 is a chart illustrating measured CO₂ for various water specimens.

### DETAILED DESCRIPTION OF THE PREFERRED EMBODIMENTS

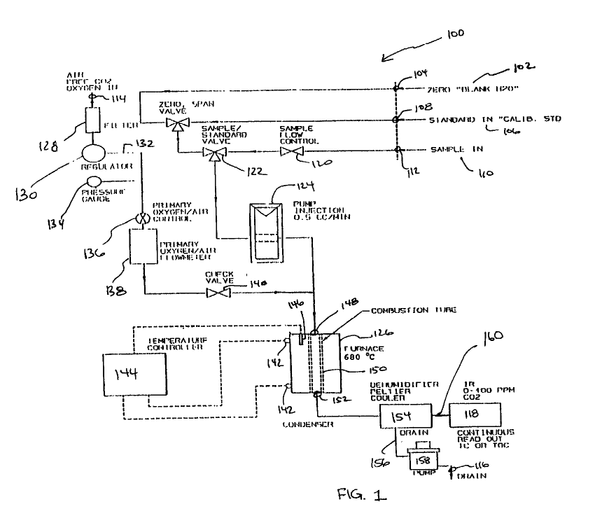

FIG. 1 is a diagrammatic view of an on-line continuous carbon analyzer for water applications in accordance with an embodiment of the present invention. System 100 receives blank water (water containing no carbon whatsoever) 102 at port 104; standard “calibration” water 106 at port 108; and a sample stream 110 at port 112. Blank water 102 and Calibration water 106 are used to determine system response to known quantities, such that systemic errors can be corrected. System 100 also receives carbon dioxide-free air at port 114. System 100 drains undesirable components out drain 116 and provides a continuous carbon readout from sensor 118.

As described above, sample 110 is provided to sample inlet port 112 and is conveyed to sample flow controller 120. Flow controller 120 is adjusted to provide a suitable flow of sample specimen to sample standard valve 122. When sample standard valve 122 is suitably actuated, sample specimen is conveyed therethrough and provided to metering pump 124. Metering pump 124 precisely controls the amount of sample specimen provided to furnace 126. Careful selection of sample flow ensures that system 100 is not overly stressed. In one preferred embodiment, pump 124 is a metering pump and controller available from Eldex Laboratories, Inc of Napa, Calif. under the trade designation Model A-60-VS. Preferably, sample flow is set to between approximately 0.5 cc per minute and approximately 2.0 cc per minute. A flow rate of 0.5 cc per minute corresponds with a controller setting of approximately 230 generating 2 to 3 drops of sample specimen per pump cycle.

Carbon dioxide-free air is received by system 100 at port 114. The gas is filtered by filter 128 and provided to pressure regulator 130. Regulator 130 is set to provide a suitable pressure on line 132 which pressure is indicated by gauge 134. The pressurized gas is conveyed to primary oxygen/air controller 136 which is adjusted to provide a suitable flow therethrough. The adjusted flow is measured by primary oxygen/air flow meter 138 and conveyed on to furnace 126 through check valve 140.

Furnace 126 is maintained at an elevated temperature, such as 680° C., in order effect high-temperature oxidation. To maintain this elevated temperature, furnace 126 is thermally coupled to heating elements 142 that are controlled by temperature controller 144 based upon a measured temperature of furnace 126 by sensor 146, which is preferably a thermocouple. Specimen 110 and pressurized gas are thus conveyed to combustion furnace 126 at furnace inlet 148. A combustion tube 150 is coupled to inlet 148 and conveys the specimen and pressurized gas to outlet 152 after it has been heated and exposed to the combustion catalyst. Preferably, tube 150 is a precision ceramic combustion tube such as that commercially available from Mindrum Precision, Inc. of Rancho Cucamonga, Calif. Within combustion tube 150, a quantity of quartz wool is preferably positioned in order to support catalyst pellets, such as platinum-based catalyst pellets. Preferably, one gram of quartz wool is disposed within combustion tube 150 as well as about 20.1 grams of catalyst pellets such as commercially available from Tekmar Company, of Cincinnati, Ohio. Additionally, 40 grams of quartz granules are also preferably positioned within combustion tube 150. The heat of combustion tube 150 as well as the catalytic materials disposed therein cause the sample to combine with oxygen and generate steam and carbon dioxide. Additional particulate matter may also be heated and conveyed from outlet 152. The heated materials are provided from outlet 152 to thermoelectric cooler 154. Preferably, thermoelectric cooler 154 employs a Peltier device generating a low temperature based upon the well-known Peltier effect. In one preferred embodiment, cooler 154 is a commercially available thermoelectric gas chiller available under the trade designation Model 600, from Universal Analyzer Inc., in Nevada. As cooler 154 cools the heated materials, water and particulate matter condense and flow into drain line 156 which is coupled to drain pump 158 to pump such materials out drain port 116. However, carbon dioxide does not flow into drain line 156, but is instead conveyed along line 160 to detector 118. Preferably, detector 118 is a known non-dispersive infrared detector that is capable of resolving 0 to 100 parts per million of CO₂. In the embodiment just described, the read-out of detector 118 will correspond with total carbon. However, those skilled in the art will recognize that organic carbon can also be measured by first conveying the sample to a solution that reacts with inorganic carbon, such as, for example, a 20% phosphoric acid solution that reacts with inorganic carbon to form carbonate and bi-carbonate. This reaction can be used to separate the inorganic carbon from the sample stream prior to analysis thereby causing detector 118 to provide an indication of total organic carbon.

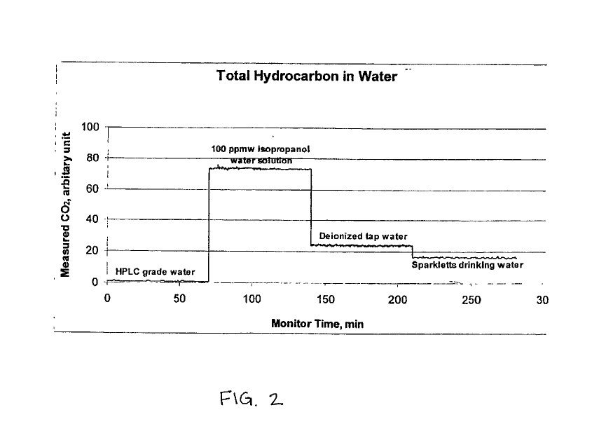

FIG. 2 is a chart of detector 118 read-out for various solutions monitored over time. As can be seen, for the first approximately 70 minutes, a solution of HPLC grade water was conveyed through system 100 providing a relatively low read-out in the range of about 2 units. Thereafter, from approximately 70 minutes to approximately 140 minutes, a solution of an isopropanol water solution of approximately 100 ppmw was conveyed through system 100 and generated a reading of approximately 75 units. Thereafter, from approximately 140 minutes to approximately 210 minutes, a solution of de-ionized tap water was conveyed through system 100 and a reading of approximately 25 units was measured. Finally, from approximately 210 minutes through 280 minutes, Sparkletts drinking water was conveyed through system 100 and generated a reading of approximately 18 units.

The system described above uses carefully selected components and component settings to generate a continuous flow that does not overly stress the system itself. Thus, the flow is small enough to inhibit excess pressure forming from the relatively significant expansion caused by heating an aqueous solution well past its boiling point. Further, providing the specimen at a relatively low temperature to a catalyst that is maintained at approximately 680° C. represents a significant thermal shock. However, the flow rates disclosed herein mitigate the thermal shock while providing suitable sample flow for useful measurements. As can be seen from the readings in FIG. 2, the output from detector 118 does not contain any large spikes that would be indicative of batch flow processing and substantial system stress.

Although the present invention has been described with reference to preferred embodiments, workers skilled in the art will recognize that changes may be made in form and detail without departing from the spirit and scope of the invention.

What is claimed is:

1. A continuous on-line carbon analyzer for water applications, the analyzer comprising:
a sample inlet to receive a continuous stream of sample specimen;
a gas inlet to receive a continuous stream of gas that is substantially free of carbon dioxide;
a sample stream flow controller coupled to the sample stream and adapted to control sample flow therethrough;
a gas flow controller coupled to the gas stream and adapted to control gas flow therethrough;
a combustion furnace coupled to the sample stream flow controller and the gas flow controller to receive continuous flow of sample stream and gas, the combustion furnace being maintained at a temperature in excess of approximately 680° C.;
a chiller coupled to the combustion furnace to receive the oxidized material and condense water and particulate matter from the heated materials; and
a detector coupled to the chiller to receive a continuous flow of carbon dioxide and provide an output indicative of a relative amount of carbon dioxide flowing therethrough.

2. The analyzer of claim 1 and further comprising a catalyst disposed in the combustion chamber.

3. The analyzer of claim 2 wherein the catalyst is a platinum-based catalyst.

4. The analyzer of claim 1 wherein the sample flow controller is a metering pump.

5. The analyzer of claim 4 wherein the metering pump provides a sample flow in the range of approximately 0.5 cc per minute to approximately 2.0 cc per minute.

6. The analyzer of claim 5 wherein the sample flow is approximately 0.5 cc per minute.

7. The analyzer of claim 1 wherein the chiller is a thermoelectric chiller.

8. The analyzer of claim 1 wherein the detector is a non-dispersive infrared detector.

9. The analyzer of claim 8 wherein the non-dispersive infrared detector provides an output corresponding to carbon dioxide quantity in the zero to 100 parts per million range.

10. The analyzer of claim 1 wherein the output is indicative of total carbon in the sample stream.

11. The analyzer of claim 1 wherein the output is indicative of the total organic carbon in the sample stream.

12. A method of continuously analyzing carbon content in water, the method comprising:
continuously receiving a sample specimen and providing the sample specimen at a pre-selected flow rate;
receiving carbon dioxide-free gas at a pre-selected flow rate;
conveying the sample stream and gas through a combustion furnace to oxidize the sample stream; and
measuring a quantity of carbon dioxide generated by the combustion furnace.

13. The method of claim 12 and further comprising cooling the oxidized sample stream prior to the step of measuring carbon dioxide quantity.

14. The method of claim 13 wherein the carbon dioxide output is indicative of total carbon in the sample stream.

15. The method of claim 12 wherein the output is indicative of total organic carbon in each sample stream.
